(19) United States
(12) Patent Application Publication
(10) Pub. No.: US 2023/0149744 A1
(43) Pub. Date: May 18, 2023

(54) THERAPEUTIC INHIBITION AND STIMULATION WITH TRANSCRANIAL ULTRASOUND

(71) Applicant: Open Water Internet Inc., San Francisco, CA (US)

(72) Inventors: Achal Singh Achrol, Highland, CA (US); Soren Konecky, Alameda, CA (US); Christopher R. Bawiec, San Francisco, CA (US); Sarah Barreto Ornellas, San Francisco, CA (US); Brad A. Hartl, San Mateo, CA (US); Ulas Taskin, Delft (NL)

(21) Appl. No.: 17/986,721

(22) Filed: Nov. 14, 2022

Related U.S. Application Data
(60) Provisional application No. 63/404,407, filed on Sep. 7, 2022.
(60) Provisional application No. 63/279,789, filed on Nov. 16, 2021.

(51) Int. Cl. A61N 7/00

(52) U.S. Cl. CPC A61N 7/00; A61N 2007/0026; A61N 2007/0078

(57) ABSTRACT

A health message is received. At least one target brain region is selected in response to receiving the health message. An ultrasound modality profile based on the health message and the at least one target brain region is generated. A wearable therapeutic ultrasound device directs the ultrasound beams to the at least one target brain region in response to the health message. The ultrasound beams are driven with the ultrasound beam profile.

### CROSS-REFERENCE TO RELATED APPLICATIONS

This application claims priority to U.S. provisional Application No. 63/279,789 filed Nov. 16, 2021 and claims priority to U.S. provisional Application No. 63/404,407 filed Sep. 7, 2022. U.S. provisional Applications 63/279,789 and 63/404,407 are hereby incorporated by reference.

### TECHNICAL FIELD

This disclosure relates generally to medical devices, and in particular to ultrasound.

### BACKGROUND INFORMATION

Noninvasive therapies are highly preferred compared to treatments that require surgery. Noninvasive treatments are even more important to therapies including the head of a patient. One noninvasive treatment is known as “Transcranial Magnetic Stimulation” (TMS). TMS is a device that can deliver stimulation to the prefrontal cortex.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 includes a system including example control components and an example wearable headset, in accordance with aspects of the disclosure.

FIGS. 2A-2C illustrate different perspectives of a therapeutic ultrasound device configured to delivered ultrasound beams to at least one target brain region of a patient/user, in accordance with aspects of the disclosure.

FIG. 3 illustrates an overhead view of a curved ultrasonic array configured to direct therapeutic ultrasound signals to targets in the brain by way of electronic beam forming, in accordance with aspects of the disclosure.

FIG. 4 shows a graphical simulation of an ultrasonic array electronically steering the collective ultrasound signal to region, in accordance with aspects of the disclosure.

FIG. 5 illustrates an ultrasonic array when all of the ultrasonic transducers in the array are emitting ultrasonic signals, in accordance with aspects of the disclosure.

FIG. 6 illustrates a system that includes processing logic driving an ultrasound modality profile onto an ultrasonic array to generate steered ultrasonic beams, in accordance with aspects of the disclosure.

FIG. 7 illustrates a flow chart of an example process of directing therapeutic ultrasound to selected brain regions, in accordance with aspects of the disclosure.

### DETAILED DESCRIPTION

Embodiments of therapeutic inhibition and stimulation with transcranial ultrasound are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects

Reference throughout this specification to “one implementation” or “an implementation” means that a particular feature, structure, or characteristic described in connection with the implementation is included in at least one implementation of the present invention. Thus, the appearances of the phrases “in one implementation” or “in an implementation” in various places throughout this specification are not necessarily all referring to the same implementation. Furthermore, the particular features, structures, or characteristics may be combined in any suitable manner in one or more implementation.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700 nm-1.6 μm.

Noninvasive transcranial neurostimulation using magnetic fields is a safe and effective treatment for episodes of major depressive disorder (MDD). In 2008, the first Transcranial Magnetic Stimulation (TMS) device was cleared by the US Food and Drug Administration (FDA) for the treatment of adult patients with treatment-resistant MDD. The original device label described a stimulation protocol targeting the left dorsolateral prefrontal cortex (DLPFC), and specified stimulation parameters that were used to establish safety and efficacy of TMS in National Institutes of Mental Health (NIMH)-supported randomized sham-controlled pivotal clinical trials.

Noninvasive brain stimulation with TMS has since been increasingly used for a growing number of research and clinical applications. Patterned repetitive TMS (rTMS) has been shown to produce long-lasting effects on neural activity and behavior beyond the stimulation period and induce neuroplastic changes. As a result, rTMS has been investigated in Alzheimer's disease (AD) and mild cognitive impairment (MCI). A recent meta-analysis was completed to systematically characterize the effectiveness of various combinations of rTMS parameters on different cognitive domains in patients with MCI and AD. A total of thirteen studies were identified comprising 293 patients with AD or MCI treated with rTMS. Results confirmed that stimulation over the left prefrontal cortex (i.e. targeting the DLPFC) significantly improved memory functions, and stimulation over the right inferior frontal gyrus (rIFG) significantly enhanced executive performance.

Transcranial focused ultrasound (tFUS) is an emerging tool for non-invasive neuromodulation that transmits low-intensity ultrasound through the skull to temporarily and safely modulate regional brain activity through acoustic wave induced mechanical-electrical effects on the neuronal membranes and ion channels. Ultrasound neuromodulation offers advantages over TMS and transcranial direct current stimulation (tDCS) that include better spatial resolution and the ability to reach deep targets in the brain. Preclinical studies have demonstrated that tFUS can reversibly modulate neuronal activity in mice, rats, sheep, pigs and monkeys with evidence of neuroplastic changes that include neurogenesis, synaptogenesis, and long term potentiation. In humans, tFUS has demonstrated the successful ability to noninvasively alter the activity of the somatosensory, visual, and thalamic brain regions. In addition, feasibility studies have demonstrated that tFUS can modulate human mood via targeting the prefrontal cortex, with associated changes in resting-state functional connectivity on magnetic resonance imaging (fMRI). These findings demonstrate that tFUS can noninvasively modulate brain function in networks related to emotional processing and mood.

Clinical applications of tFUS for neuromodulation (either stimulation or inhibition) include the treatment of psychiatric and neurological disease, including MDD, Generalized Anxiety Disorder (GAD), Obsessive-Compulsive Disorder (OCD), Substance Use Disorder (SUD, and other Addictions), Post-Traumatic Stress Disorder (PTSD), Alzheimer's Disease (AD), Parkinson's Disease, and other forms of cognitive dysfunction.

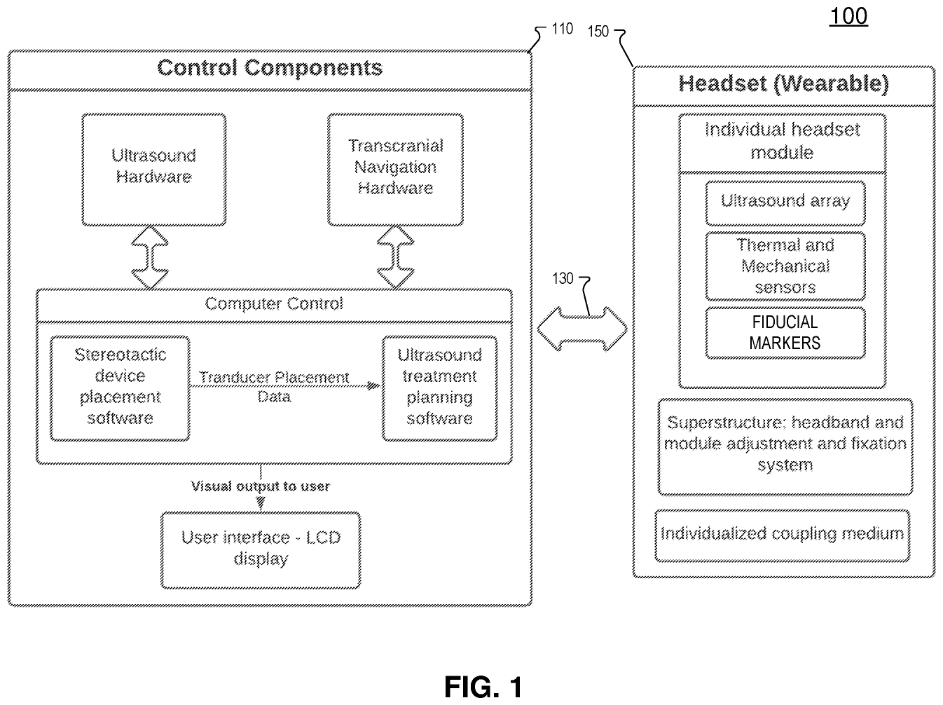

Implementations of the disclosure include a wearable device and systems that support a wearable device. The device may be worn on or about the head, for example. Implementations may include a phased array ultrasound transducer worn on the forehead to transmit therapeutic ultrasound doses to the frontal lobe, or other targeted brain regions, for neuromodulation applications. In some implementations, a multi-element phased array ultrasound transducer is disposed along a curvature. A Block diagram schematic of an example system is included in FIG. 1.

FIG. 1 includes a system 100 including example control components 110 and an example wearable headset 150, in accordance with aspects of the disclosure. A cable 130 carrying power and data is coupled between headset 150 and control components 110. In some implementations of system 100, cable 130 is absent and wearable headset 150 relies on wireless data and battery power. In some implementations, a wearable headset includes all of the components in control components 110.

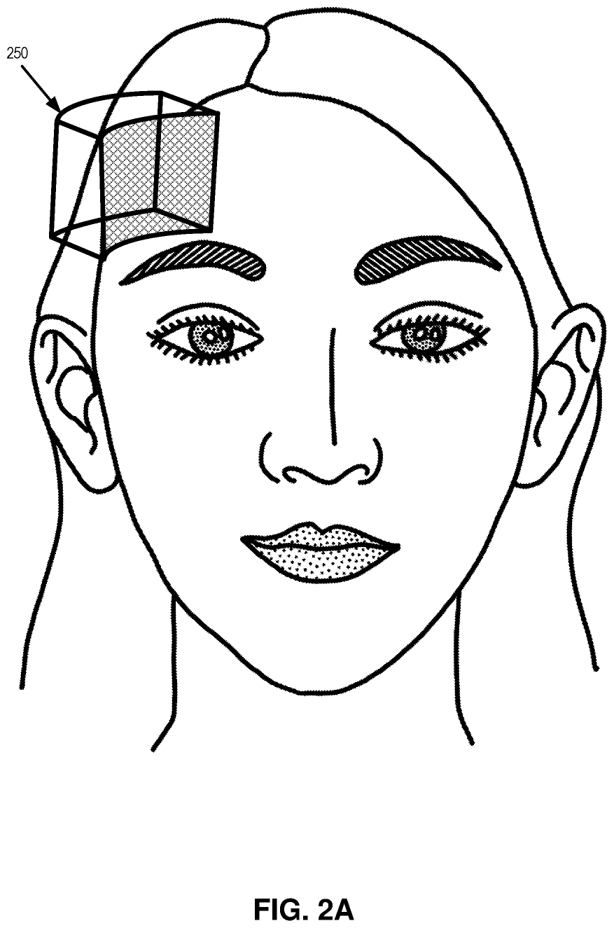

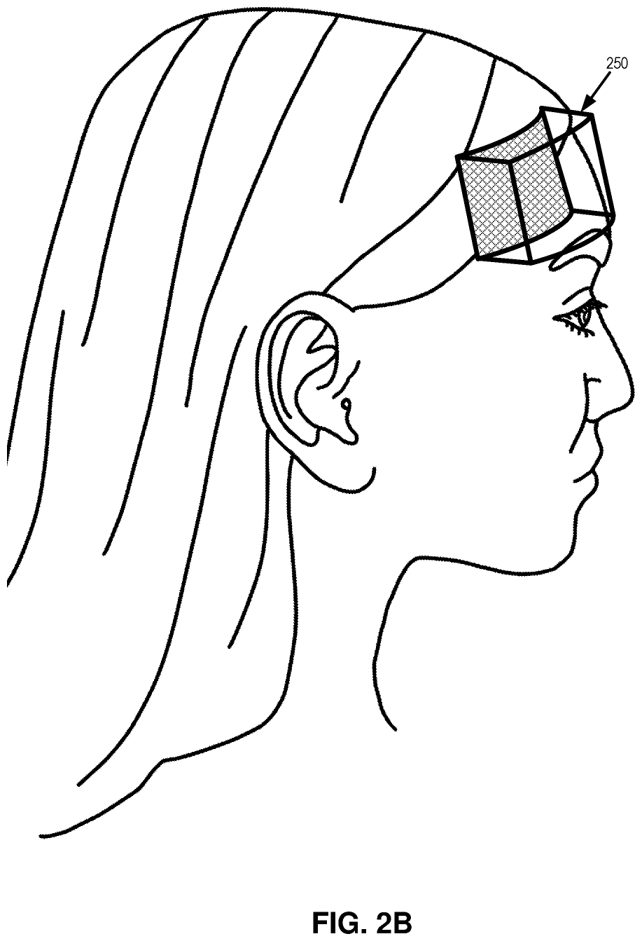

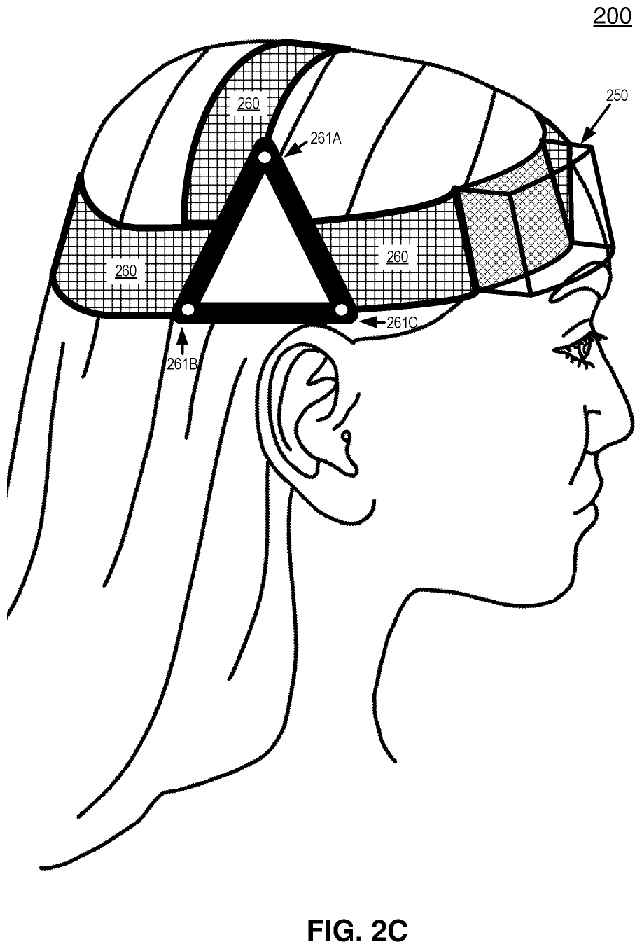

FIGS. 2A-2C illustrate different perspectives of a therapeutic ultrasound device 250 configured to delivered ultrasound beams to at least one target brain region of a patient/user, in accordance with aspects of the disclosure. FIG. 2A illustrates a therapeutic ultrasound device 250 may be sized and configured to conform to a head of a patient/user. The crosshatch fill of therapeutic ultrasound device 250 may be a curvature that ultrasonic transducers are disposed on in order to deliver steered ultrasound beams to the target brain region. The target brain regions may include a prefrontal cortex, a medial prefrontal cortex, or an anterior medial prefrontal cortex, for example. FIG. 2B illustrates a side view of therapeutic ultrasound device 250 on a head of a patient.

FIG. 2C illustrates a side view of a wearable 200 that includes therapeutic ultrasound device 250, securing apparatus 260, and fiducial markers 261A, 261B, and 261C (collectively referred to as fiducial markers 261), in accordance with aspects of the disclosure. Fiducial markers 261 assist in identifying the position of therapeutic ultrasound device 250 with respect to the user. Fiducial markers 261 may be small metal (e.g. gold) spheres, coils, or cylinders that assist in guiding the ultrasound beams from therapeutic ultrasound device 250 to the specified brain region. Securing apparatus 260 holds and secures fiducial markers 261 and therapeutic ultrasound device 250 to the head of the patient. Securing apparatus 260 may include a strap that includes elastic fabric and/or fasteners to tighten and secure the elastic fabric.

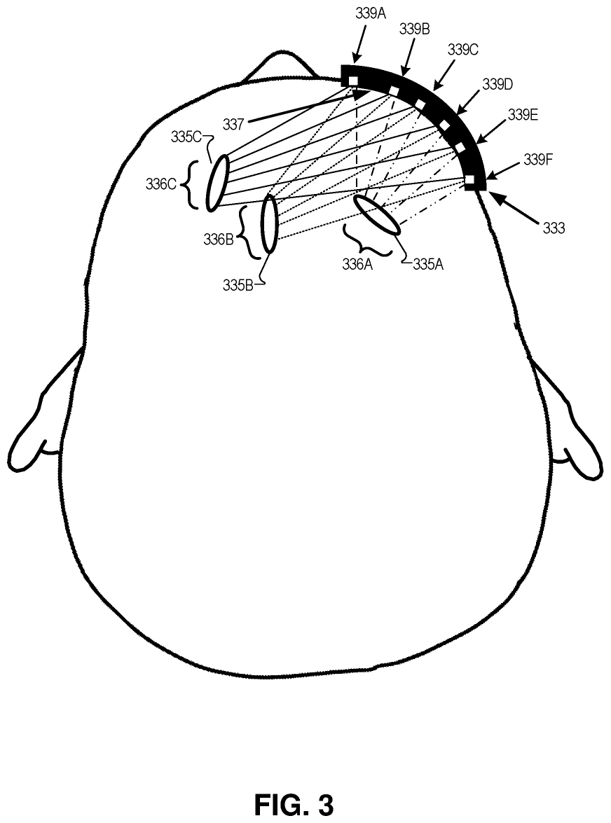

FIG. 3 illustrates an overhead view of curved ultrasonic array 333 configured to direct therapeutic ultrasound signals to target regions 335 in the brain by way of electronic beam forming, in accordance with aspects of the disclosure. Ultrasonic array 333 may include individually-addressable ultrasonic transducers. There may be 16, 32, 64, 128, 256, or 1024 ultrasonic transducers in the ultrasonic array 333, for example. In the illustration of FIG. 3, ultrasonic transducers 339A, 339B, 339C, 339D, 339E, and 339F, are illustrated. In some implementations, the ultrasonic transducers are disposed on a two-dimensional grid having x columns and y rows. The ultrasonic transducers of curved ultrasonic array 333 are disposed on a curvature 337 in FIG. 3. Curvature 337 may be cylindrical. In some implementations, the ultrasonic transducers of curved ultrasonic array 333 are disposed on a spherical curvature. In some implementations, the ultrasonic transducers of curved ultrasonic array 333 are disposed on an aspherical curvature. Disposing the ultrasonic transducers on a curved surface may assist in fitting the device to the tissue/forehead of the patient. Disposing the ultrasonic transducers on a curved surface may also assist in beam forming the ultrasound signals to reach the perimeters of the tissue, whereas ultrasonic transducers on a planar surface may be limited to reaching only certain portions of the tissue.

Processing logic may drive the ultrasonic transducers to a beam shape that electronically steers the ultrasound signals to different points in the brain. The processing logic may be included in wearable 150/200 and/or in control components 110. The beam shaping may rely on constructive and/or destructive interference of the individual ultrasound signals from individual ultrasound transducers to shape the collective ultrasound signal. Processing logic may drive ultrasonic array 333 to “focus” on region 335A by beam shaping the collective ultrasonic signal 336A (dot-dash-dash lines) to be directed to region 335A. The processing logic may also drive ultrasonic array 333 to “focus” on region 335B by beam shaping the collective ultrasonic signal 336B (dotted lines) to be directed to region 335B or to “focus” on region 335C by beam shaping the collective ultrasonic signal 336C (solid lines) to be directed to region 335C. The collective ultrasonic signals may be focused on different regions during a treatment plan. The treatment area of different portions of the brain may vary from patient to patient and depend on the particular therapy.

Electronic beam steering using the plurality of ultrasonic transducers in ultrasonic array 333 may be advantageous over prior solutions that rely on mechanical beam steering (e.g. physical rotation of the device with respect to the patient). Electronic beam steering using the plurality of ultrasonic transducers in ultrasonic array 333 may be advantageous over prior solutions or devices that relied on selecting/activating a particular individual transducer in the array to change the direction/location of the ultrasonic signal. In these prior solution, the particular individual transducer was selected to deliver the ultrasound signal based on the particular transducer's mechanical spacing and/or proximity to the region rather than relying on destructive and/or constructive interference of ultrasonic signals from a plurality of ultrasonic transducers. Electronic beam steering using the plurality of ultrasonic transducers in ultrasonic array 333 may be advantageous to “sharpen” and “soften” focus of the collective ultrasound signal, based on the goals of the particular medical therapy.

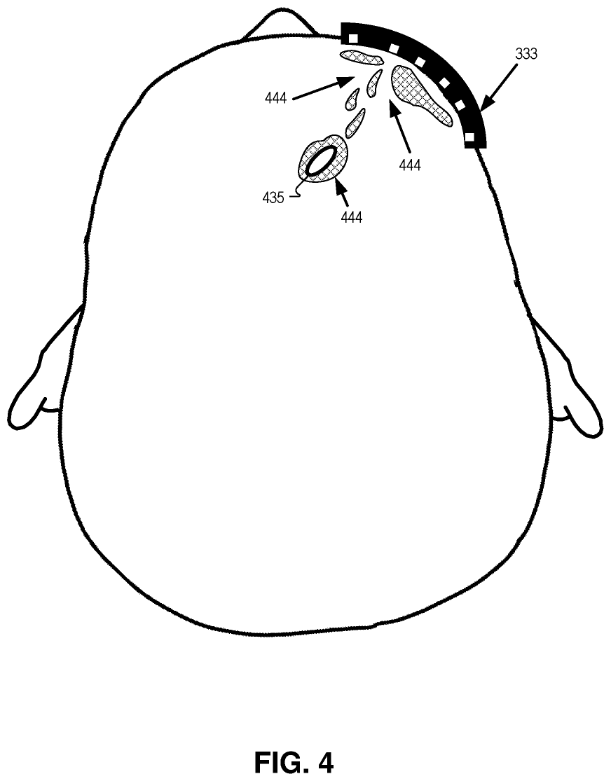

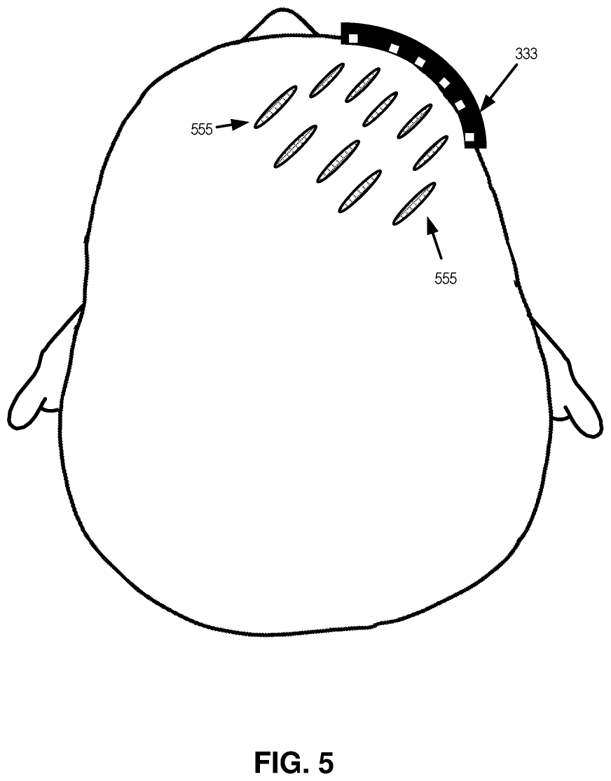

FIG. 4 shows a graphical simulation of ultrasonic array 333 electronically steering the collective ultrasound signal 444 to region 435. Thus, FIG. 4 illustrates that ultrasonic array 333 can be driven to steer ultrasound signal 444 to “focus” on a particular region 435. Ultrasound signal 444 is illustrated as crosshatch-filled irregular shapes in FIG. 4. In contrast to FIG. 4, FIG. 5 illustrates ultrasonic array 333 when all of the ultrasonic transducers in the array are emitting ultrasonic signals 555 simultaneously. In other words, the ultrasonic array 333 in FIG. 5 is not being driven for beam forming or electronic steering of the ultrasonic signal 555. Hence, the ultrasonic signal 555 is not focused to a particular portion of tissue in FIG. 5.

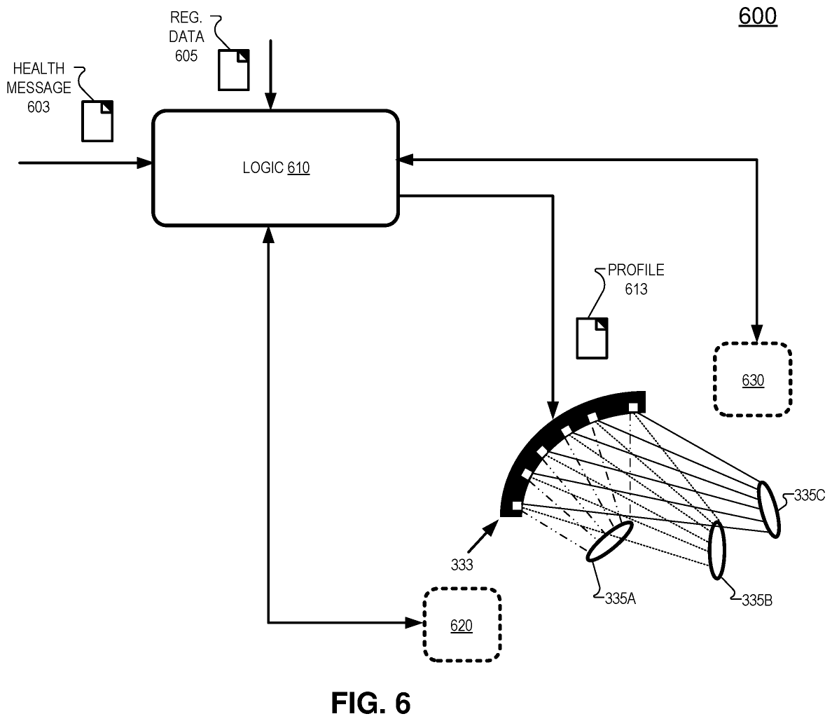

FIG. 6 illustrates a system 600 that includes processing logic 610 driving ultrasound modality profile 613 onto ultrasonic array 333 to generate steered ultrasonic beams, in accordance with aspects of the disclosure. System 600 may be included in wearable 150 or 200, for example. Processing logic 610 receives a health message 603. Health message 603 may include digital bits. Health message 603 may be provided by control components 110 by wireless or wired communication channel. Health message 603 may include a symptom indicator or a diagnosis indicator of a patient. The diagnosis indicator may be a diagnostic code provided by, or prescribed by, a physician.

The diagnosis indicator may specify a psychiatric and/or neurological disease, including NIDD, Generalized Anxiety Disorder (GAD), Obsessive-Compulsive Disorder (OCD), Substance Use Disorder (SUD, and other Addictions). Post-Traumatic Stress Disorder (PTSD), Alzheimer's Disease (AD) Parkinson's Disease, and other forms of cognitive dysfunction. The health message 603 may indicate symptoms relating to Suicidal Ideation, Perseverative Negative Thinking/Rumination, Anxious Avoidance, Negative Bias, Threat Dysregulation, Anhedonia, Context Insensitivity, Inattention, Cognitive Control Dysfunction, management of additive Cravings or Triggers, improvement of cognitive function/memory function, enhancing executive performance, or the management of other acute forms of symptomatic mental thought content.

Processing logic 610 may be configured to select at least one target brain region (e.g. regions 335A, 335B, or 335C) in response to receiving health message 603. Therapies for different symptoms or medical conditions may be best treated by targeting different portions of the brain, for example.

Processing logic 610 may be further configured to generate an ultrasound modality profile 613 based on (1) health message 603; and (2) the at least one target brain region. In some implementations, processing logic 610 is configured to generate ultrasound modality profile based on (1) health message 603; (2) the at least one target brain region; and (3) patient anatomy registration data 605. Patient anatomy registration data 605 may include medical imaging such as a Computed Tomography (CT) or Magnetic Resonance Imaging (MRI) of the tissue that will receive the steered ultrasonic beams from ultrasonic array 333. The medical imaging of the tissue may include fiducial markers 261. In an implementation, the patient anatomy registration data 605 is generated by using an optical or electromagnetic registration technique.

Processing logic 610 may be further configured to direct ultrasound beams to the at least one target region in response to receiving the health message 603. The ultrasound beams (e.g. 336A, 336B, or 336C) are shaped according to the ultrasound modality profile 613 that are driven onto ultrasonic array 333 by processing logic 610. In some implementations, processing logic 610 is configured to direct ultrasound beams to more than one target region 335 in response to health message 603.

In some implementations, thermal and mechanical sensors 620 in system 600 may be included in wearable 150 or 200 to monitor the patient for thermal and mechanical signals while the ultrasonic therapy is in process. The processing logic (e.g. processing logic 610) may adjust the collective ultrasound signal (power and/or location) in response to the thermal and mechanical signals. The processing logic may pause/stop the collective ultrasound signal (power and/or location) in response to the thermal and mechanical signals. Thermal sensors may include thermometers. Mechanical sensors may include cavitation sensors.

In addition to beam forming, processing logic 610 may also adjust the ultrasonic power generated by ultrasonic array 333. In an implementation, the ultrasonic power is adjusted based on the skull properties (thickness, density, and speed of sound) of the patient. In other words, processing logic adjusts driving the ultrasonic sonic transducers in ultrasonic array 333 in response to skull properties (thickness, density, and speed of sound) received in the patient registration data 605. The skull properties may be derived from medical imaging or by measurements taken by wearable 150 or 200.

In an implementation, processing logic 610 adjusts the ultrasound modality profile 614 for driving the ultrasonic transducers in ultrasonic array 333 in response to an optical measurement of the patient's brain performed by optical device 630. Optical measurements of the brain may generate blood flow data, blood volume data, and/or blood oxygenation data. The optical measurements may include illuminating the tissue with laser light and measuring speckle in the return signal, for example. Thus, optional optical device 630 may include a laser configured to illuminate the tissue and a photodetector such as a photodiode, array of photodiodes, or an image sensor to measure the returning light.

In some implementations, the neuromodulation techniques of the disclosure are delivered based on a specific disease or a specific target symptom. Depending on the disease or symptom, particular brain regions may be targeted to treat the disease or symptom. Furthermore, different ultrasound modality profiles may be preferred for treating different diseases and symptoms. For example, a stimulating ultrasound profile or an inhibition ultrasound profile may be advantageous for providing therapy for different diseases/symptoms. The difference between stimulating ultrasound profiles and inhibition ultrasound profiles may be the power, duration, duty cycle, frequency, and repetition, among other aspects. Different ultrasound profiles will be described in more detail below.

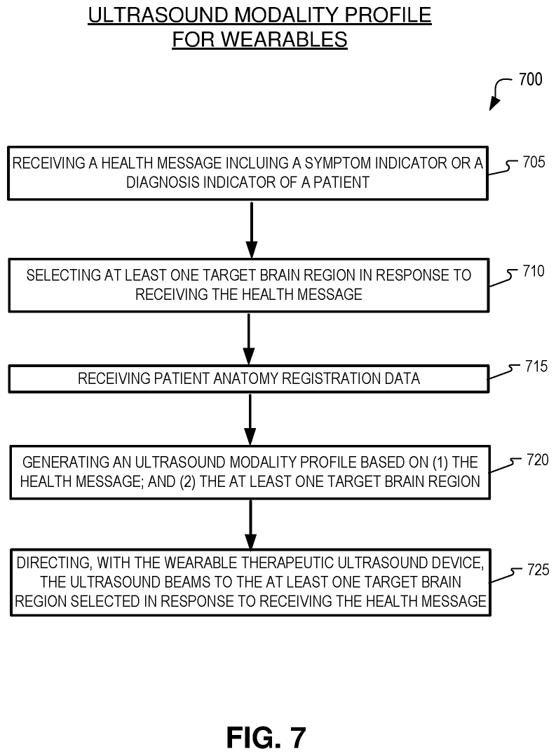

FIG. 7 illustrates a flow chart of an example process 700 of directing therapeutic ultrasound to selected brain regions, in accordance with aspects of the disclosure. The order in which some or all of the process blocks appear in process 700 should not be deemed limiting. Rather, one of ordinary skill in the art having the benefit of the present disclosure will understand that some of the process blocks may be executed in a variety of orders not illustrated, or even in parallel. Processing logic included in a therapeutic ultrasound wearable, a smartphone, tablet, or computer may execute all or a portion of process 700.

In process block 705, a health message is received that includes a symptom indicator or a diagnosis indicator or a patient. The health message may be received by way of a touch-screen user interface input on a smartphone or tablet, for example, although the health message may be received via any suitable user input interface. The health message may also be received by way of wired or wired transmission from a computer or remote server (a.k.a. “the cloud”).

In process block 710, at least one target brain region is selected in response to receiving the health message. In some implementations, a brain network that includes multiple brain regions may be selected in response to receiving the health message.

In process block 715, patient anatomy registration data is received. The patient anatomy registration data registers a brain of the patient in space with respect to a wearable therapeutic ultrasound device (e.g. device 250). In an implementation, the patient anatomy registration data includes the co-registration of a medical image (of the patient's brain) and the placement of the wearable, as described above.

In process block 720, an ultrasound modality profile is generated based on (1) the health message; and (2) the at least one target brain region. In some implementations, the ultrasound modality profile is also generated based on the patient anatomy registration data.

Example ultrasound modality profiles may include the following characteristics: (A) Carrier frequency (50 kHz to 1 MHz, 200 kHz to 600 kHz, or 350 kHz to 450 kHz), (B) burst width (50 us to 1 sec, 100 us to 500 ms, or 500 us to 5 ms) (C) pulse repetition frequency (1 Hz to 5 kHz, 5 Hz to 1 kHz, or 10 Hz to 250 Hz) (D) duty cycle (0.01% to 100%, 1% to 80%, or 2%-10%), (E) sonication pattern (10 us to 120 seconds of bursts followed by stopping or resting for 10 us to 300 seconds and repeating, or more narrowly 100 us to 60 sec seconds of bursts followed by stopping or resting for 100 us to 60 seconds and repeating or burst repetition period, or even more narrowly 1 ms to 30 sec seconds of bursts followed by stopping or resting for 1 ms to 30 seconds and repeating). This bursting followed by resting can carry on for up to for as little as one second, but up to 30 minutes, typically being between 30 seconds and 10 minutes.

In an implementation, the ultrasound modality profile is a stimulation ultrasound profile. The stimulation ultrasound profile may have a duty cycle of 5% or less. The stimulation ultrasound profile may have a duration of 1 minute or less. In an implementation, the stimulation ultrasound profile includes approximately a burst width of 2 ms, a burst repetition frequency of 250 Hz, a 50% duty cycle repeated for 500 ms followed by 500 ms off time for a total duration of 10 seconds.

In an implementation, the ultrasound modality profile is an inhibition ultrasound profile. The inhibition ultrasound profile may have a duty cycle of greater than 5%. The stimulation ultrasound profile may have a duration between 1 minute and 10 minutes. In an implementation, the inhibition ultrasound profile includes approximately 5 ms burst width, burst repetition frequency of 10 Hz, a 5% duty cycle repeated for 30 seconds, then resting for 30 seconds and repeating for up to 30 minutes.

In process block 725, ultrasounds beams are directed, with the wearable therapeutic ultrasound device, to the at least one target brain region selected in response to the receiving the health message. The ultrasound beams are driven with the ultrasound modality profile generated in process block 720.

In an implementation, the target brain region is the dorsolateral prefrontal cortex. In an implementation, the target brain region is the cingulate cortex. In an implementation, the target brain region is the anterior cingulate cortex. In an implementation, the target brain region is the anterior medial prefrontal cortex when the health message includes a Major Depressive Disorder (MDD) indicator. In an implementation, the target brain region is the anterior medial prefrontal cortex when the health message includes a Generalized Anxiety Disorder (GAD) indicator. In an implementation, the target brain region is the anterior medial prefrontal cortex when the health message includes a Perseverative Negative Thinking or Rumination indicator.

In an implementation, the target brain region is the anterior cingulate cortex when the health message includes an indicator of Substance Use Disorder (SUD) or other addictive disorders, including drug abuse, alcohol abuse, smoking cessation, gambling, pornography or internet addiction disorders. In an implementation, the target brain region is the anterior cingulate cortex when the health message includes an indicator of Anxious Avoidance, Negative Bias, Context Insensitivity. In an implementation, the target brain region is the anterior cingulate cortex and/or medial prefrontal cortex, including part or all of the dorsal medial prefrontal cortex, dorsal anterior cingulate cortex, and ventral medial prefrontal cortex, for the purpose of treating symptoms relating to Threat Dysregulation or Anhedonia indicators included in the health message.

In an implementation, the target brain region is the Orbitofrontal Cortex (OFC) for the purpose of treating Obsessive-Compulsive Disorders (OCD) indicators included in the health message. In an implementation, the target brain region is the Orbitofrontal Cortex (OFC) for the purpose of treating Obsessive-Compulsive Disorders (OCD) indicators included in the health message. In an implementation, the target brain region is the OFC for the purpose of treating symptoms of anhedonia indicators included in the health message. In an implementation, the target brain region is the insula and/or amygdala for the purpose of treating negative bias or threat dysregulation indicators included in the health message. In an implementation, the target brain region is the insula and/or amygdala for the purpose of treating Post-Traumatic Stress Disorder indicators included in the health message. In an implementation, the target brain region is selected from either the dorsolateral prefrontal cortex, inferior frontal gyms, or mesial temporal lobe including entorhinal or hippocampus when Alzheimer's Disease indicators are included in the health message.

In another example process of the disclosure: (1) a medical image of the patient/user's head is acquired. The medical image may include imaging of fiducial markers 261. The medical image may be an CT or MRI, for example. Subsequently, (2) the medical image and the placement of therapeutic ultrasound device 250 are co-registered. This co-registration may include a stereotactic optical technique. (3) Patient baseline neurological function is acquired. (4) A patient treatment plan is created that defines one or more target regions of sonification of the patient-specific anatomy (e.g. registered MR/CT) and an ultrasound dose is defined (beam steering, acoustic parameters). (5) The patient treatment plan is performed on the patient. A follow-up neurological assessment may subsequently be acquired for clinical documentation and efficacy.

In an example implementation of the disclosure, a wearable ultrasound transducer array is designed specifically for transcranial targeting through the forehead and have one or more of the following features: (1) positioned on the forehead; (2) electronic steering of individual transducers within the array to the frontal lobe or other specific target brain regions; (3) targeting the specific brain regions with an intentionally-shaped focus using stereotactic guidance to patient-specific anatomy.

In an example implementation of the disclosure, a curved array is configured to optimally target underlying brain regions from the forehead and temple with a wearable design. In some implementations, the array is not necessarily curved.

In an example implementation of the disclosure, the method for positioning the ultrasound array includes using anatomical landmarks. In an implementation, a broadband ultrasound transducer array is used so that the frequency can be adjusted based on application and the ultrasound modality profile for customized treatment plans.

The term “processing logic” (e.g. processing logic) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Communication channels may include or be routed through one or more wired or wireless communication utilizing IEEE 802.11 protocols, BlueTooth, SPI (Serial Peripheral Interface), I²C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a smartwatch, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

The tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A method comprising:
receiving a health message, wherein the health message includes a symptom indicator or a diagnosis indicator of a patient;
selecting at least one target brain region in response to receiving the health message;
receiving patient anatomy registration data, wherein the patient anatomy registration data registers a brain of the patient in space with respect to a wearable therapeutic ultrasound device;
generating an ultrasound modality profile based on (1) the health message; and (2) the at least one target brain region; and
directing, with the wearable therapeutic ultrasound device, ultrasound beams to the at least one target brain region selected in response to receiving the health message, wherein the ultrasound beams are driven with the ultrasound modality profile.

2. The method of claim 1, wherein the ultrasound modality profile is an inhibition ultrasound profile.

3. The method of claim 2, wherein the inhibition ultrasound profile includes approximately 5 ms burst width, burst repetition frequency of 10 Hz, a 5% duty cycle repeated for 30 seconds, then resting for 30 seconds and repeating for up to 30 minutes.

4. The method of claim 2, wherein the inhibition ultrasound profile has a duty cycle greater than 5%.

5. The method of claim 2, wherein the inhibition ultrasound profile has a duration between 1 minute and 10 minutes.

6. The method of claim 1, wherein the ultrasound modality profile is a stimulation ultrasound profile.

7. The method of claim 6, wherein the stimulation ultrasound profile includes approximately a burst width of 2 ms, a burst repetition frequency of 250 Hz, a 50% duty cycle repeated for 500 ms followed by 500 ms off time for a total duration of 10 seconds.

8. The method of claim 6, wherein the stimulation ultrasound profile has a duty cycle less than 5%.

9. The method of claim 6, wherein the stimulation ultrasound profile has a duration less than 1 minute.

10. The method of claim 1, wherein the generating the ultrasound modality profile is also based on the patient anatomy registration data.

11. The method of claim 1, wherein the ultrasound beams are generated by a phased array ultrasound transducers.

12. The method of claim 11, wherein the phased array ultrasound transducers are disposed along a curved surface of a head mounted wearable.

13. The method of claim 1, wherein the at least one brain region includes a frontal lobe or a prefrontal cortex.

14. A wearable device comprising:
an array of ultrasound transducers disposed along a curvature sized to conform to a head of a patient;
processing logic configured to:
receive a health message, wherein the health message includes a symptom indicator or a diagnosis indicator of the patient;
select at least one target brain region in response to receiving the health message;
generate an ultrasound modality profile based on (1) the health message; and (2) the at least one target brain region; and
direct ultrasound beams to the at least one target brain region selected in response to receiving the health message, the ultrasound beams driven with the ultrasound modality profile.

15. The wearable device of claim 14, wherein the ultrasound modality profile is an inhibition ultrasound profile.

16. The wearable device of claim 15, wherein the inhibition ultrasound profile has a duty cycle greater than 5%.

17. The wearable device of claim 15, wherein the inhibition ultrasound profile has a duration between 1 minute and 10 minutes.

18. The wearable device of claim 14, wherein the ultrasound modality profile is a stimulation ultrasound profile.

19. The wearable device of claim 18, wherein the stimulation ultrasound profile includes approximately a burst width of 2 ms, a burst repetition frequency of 250 Hz, a 50% duty cycle repeated for 500 ms followed by 500 ms off time for a total duration of 10 seconds.

20. The wearable device of claim 14, wherein the generating the ultrasound modality profile is also based on patient anatomy registration data.
